(19) United States
(12) Patent Application Publication
(10) Pub. No.: US 2017/0232048 A1
(43) Pub. Date: Aug. 17, 2017

(54) Adherent Dental Synbiotic Lozenge for Oral and General Health

(71) Applicant: RENUZORAL LLC, LAGUNA NIGUEL, CA (US)

(72) Inventor: STEVEN J. EDWARDS, LAGUNA NIGUEL, CA (US)

(21) Appl. No.: 15/436,606

(22) Filed: Feb. 17, 2017

Related U.S. Application Data
(60) Provisional application No. 62/296,526, filed on Feb. 17, 2016.

(51) Int. Cl. A61K 35/747; A61K 9/00; A61K 35/744; A61K 8/99; A61K 8/73; A61K 31/733; A61K 8/97; A61K 36/48; A61K 31/715; A61K 31/723; A61K 47/26; A61K 8/60; A61K 47/38; A61K 47/36; A61K 47/10; A61K 8/34; A61K 47/02; A61K 8/24; A61K 47/46; A61K 47/12; A61K 8/36; A61Q 11/00; A61K 8/02

(52) U.S. Cl. CPC A61K 35/747; A61K 8/0233; A61K 9/0056; A61K 35/744; A61K 8/99; A61K 8/73; A61K 31/733; A61K 8/97; A61K 36/48; A61K 31/715; A61K 31/723; A61K 47/26; A61K 8/60; A61K 47/38; A61K 8/731; A61K 47/36; A61K 8/733; A61K 47/10; A61K 8/345; A61K 47/02; A61K 8/24; A61K 47/46; A61K 47/12; A61K 8/36; A61Q 11/00; A61K 2035/11

(57) ABSTRACT

Provided is a more effective dental synbiotic lozenge that is intra-orally adhesive. The lozenge contains adhesive prebiotics and excipients, and one or more species of probiotic organisms. While safely adhered to an intra-oral surface, the lozenge dissolves to provide a controlled time-release of the excipients, prebiotics, and probiotic organisms over a period of hours instead of minutes. The lozenge dissolves into a malleable biofilm that includes a natural biofilm formed by the prebiotics and a supplemental biofilm formed by the excipients. The lozenge can be used while sleeping, which can increase probiotic availability exponentially. The result is a lozenge that allows for a smaller, but more diverse, microbial payload that can grow over time such that a fewer number of organisms are needed from the start. In one embodiment, the lozenge contains adhesive prebiotics acacia and inulin, along with additional excipients microcrystalline cellulose, HPMC K15, and sodium alginate.

### PRIORITY CLAIM

This applications claims the priority date of provisional application No. 62/296,526 filed on Feb. 17, 2016, which is herein incorporated by reference in its entirety.

### FIELD OF THE INVENTION

The present invention is in the field of probiotics, and more particularly, dental synbiotic lozenges.

### BACKGROUND

Current dental probiotics come in various delivery system forms and formats. They include probiotic-coated straws, drops, tablets, lozenges, powders, strips, liquids, capsules, toothpastes, oils, and chewing gums. However, given the nature of the mouth, delivering and growing dental probiotics with safe, effective, extended duration is much different from delivering intestinal probiotics. A major problem with the aforementioned delivery systems is that none of them have been designed for probiotic adhesion, substantivity, nutrition, protection, growth, and replication in the mouth. Although saliva is mostly water, it also contains many enzymes and immunoglobulins to attack unprotected microorganisms. Thus, the probiotic organisms are merely released into a hostile oral environment without adequate nutritional support and protection. Due to salivary flow, chewing, tongue movement, swallowing and other oral activities, the probiotic dose is mostly gone from the mouth within a few seconds to about 15 minutes. Once the probiotic leaves the mouth, any beneficial dental effects are lost. What is needed is a method of probiotic administration that mimics the natural way that microbes colonize the oral region.

But most importantly, because probiotics are freeze-dried organisms, they must be rehydrated and undergo critical metabolic processes before they can grow and colonize their niches. This process takes much longer than 15 minutes, which is the amount of time a conventional dental lozenge lasts in the mouth before being rendered ineffective by chewing and/or swallowing. As a result, conventional probiotic dental lozenges are not effective.

### Conventional Probiotic Lozenge Manufacture, Performance, and Use

Hundreds of billions of probiotic organisms are placed in growth media within incubator vats, but the organisms do not grow immediately. They first go through a lag phase which can take up to 2.5 hours. During this time the organisms are adapting to their environment, activating or deactivating various genes, and repairing membranes and intracellular machinery. They are only growing larger and preparing for the start of the logarithmic phase, which is exponential replication in great numbers. The logarithmic phase is when the microbes replicate exponentially. After the logarithmic phase, the organisms reach a steady phase during which they have no more room for growth. If left too long in this phase, they will begin to die off and decrease in number until only a hardy few survive. At some time during the steady phase, their colonies are skimmed, collected, freeze-dried, desiccated, and crumbled into powder, which essentially puts them into “hibernation” for lozenge manufacture. Their cell membranes are crinkled and often cracked, with their internal machinery is jumbled, yet most can survive this step and eventually be reconstituted.

Since the organisms destined for dental use will not need to survive the harsh journey through acids in the stomach and enzymes in the small intestine, they are not artificially encapsulated after growth because they would never be available for use in the oral cavity. During lozenge manufacture, billions of organisms are mixed with tableting excipients, flavorings, etc., and smashed into tablet-making presses. The heat and pressure of smashing the probiotics into tablet or lozenge shapes kills some 15% of the probiotics immediately. Thus, some 3.529+billion organisms must be used at outset to provide, say, 3 billion viable organisms at time of packaging. During packaging, lozenges are dispensed into bottles, and desiccant packs with oxygen scavengers are added to the bottles to absorb potential seal leaks and humidity that might condense within the bottles.

During storage, some organisms die if they become moisturized and “re-awakened” but have no food to survive on. Even if the probiotic bottle is never opened, the microorganisms slowly die off at an average rate such that after 18 months from manufacture, approximately 33%, or, one billion organisms, will still be viable. Therefore, approximately 111 million organisms die off in the bottle every month. Then the organisms must withstand the rigors of storage, shipping to warehouse or retail store, temperature fluctuations, re-storage, re-shipping or purchase and carry-home, then more storage at home with repeated container openings and exposures to air, humidity, and contamination. More organisms die during this extended phase. If the organisms have been tableted along with their preferred prebiotic food, and they “wake up”, they have a better chance of survival during this arduous time from manufacture to use.

During the home storage, when the product is actually used, a person opens a probiotic package or bottle and puts a typical ordinary probiotic tablet in their mouth. Various events occur during this period of use and storage. The desiccant/scavenger packs cannot absorb all the oxygen and moisture every time the bottle is opened, so, some of the organisms begin to re-awaken, grow a little, and slowly resume their life cycles within the bottle, but without food they eventually die inside the bottles. Even with food they can die, albeit much more slowly. Hence the count of organisms listed on the bottle label only matters before opening the bottle. It is also why organisms should be tableted along with their preferred foods, because after opening, the organism count becomes uncertain, especially if they have no food. This is also why probiotics usually come in 30-count bottles (and not higher counts) so that consumers are forced to run out of supply fairly quickly before the organisms die off massively.

With a conventional probiotic lozenge, as the lozenge begins dissolving in the mouth, the excipients merely dissociate away and the organisms freely float around in saliva as planktonic organisms and are swallowed. The person unconsciously moves the lozenge around in the mouth and speeds up the dissolution, or chews it and swallows it almost immediately. Thus, most of the organisms in a typical lozenge are swallowed and are gone from the mouth in about 15 minutes. Few of the organisms are able to find their niches, let alone adhere to their niches and begin colonizing and populating. This is one reason conventional dental probiotics must contain many billions of organisms—because most of them simply die and are diluted and swallowed into the digestive system where they do no good for the mouth. People are told to let the tablets dissolve slowly, but most people end up chewing and swallowing them too soon. Therefore, many billions of organisms must be used at the outset in order to ensure that enough might be left behind after many die and most of the rest are merely swallowed. Also, people use probiotic tablets during waking hours because it may be dangerous to sleep with a loose tablet floating about in the mouth. Even an adherent tablet that remains as a discrete tablet and does not transform into a film may have safety concerns.

This conventional approach is inefficient, haphazard, inconsistent, wasteful, expensive, and especially limiting in terms of types of organisms to be used and quantity required. Because billions of organisms are needed at the start, it is expensive to use more than a few types of organisms, thus limiting the choices of strains and blocking the potential synergistic effects of having organism diversity. In the microbial world, diversity is desirable because microbes are communal and work best in groups. Diversity is also essential for oral heath because of its numerous types of niches and the vast conditions to which the oral cavity is exposed. But because of needing so many billions at the outset, the concept of using potentially synergistic combinations of various types of organisms is prohibitively expensive. As a result, most dental probiotics are limited to just one or a few types of organisms. Furthermore, if the sleeping hours could be utilized for probiotic delivery, the organisms would have an exponentially greater chance for growth, colonization, and population.

Upon a tablet or lozenge entering the mouth, the organisms begin rehydrating. Rehydration of organisms requires up to 5 minutes if a lozenge is left to dissolve on its own. However, rehydration might require 2 minutes if the tablet is chewed and all the particles are exposed to saliva. Yet when tablets are chewed, they are usually swallowed sooner than 2 minutes. Furthermore, most typical dental probiotic lozenge excipients tend to be slightly gritty or chalky and provide an unpleasant mouth feel. Therefore, most people swallow such lozenge particulates sooner than later, just to rid the unpleasant sensation from the mouth. Lozenges that are manufactured with fast-melt technology have a smoother, creamier texture and nicer mouth feel, but their rapid dissolution and rapid swallowing defeats the purpose of trying to rehydrate and grow microbes and colonize the mouth. The time elapsed at this point of total dissolution is 2 to 5 minutes.

Once the organisms are rehydrated, they need to recognize and adapt to their new environment. Adaptation could require from 4 minutes to 20 minutes. At this point the elapsed time may be 6 minutes to 25 minutes. However, most typical probiotic lozenges would already be swallowed or mostly gone by 15 minutes. After adaptation, the organisms do not immediately “grow” as they must repair damaged systems, turn on or off certain genes, avoid predators, toxins, enzymes, and immunoglobulins, find food, grow larger (not yet in numbers) and prepare themselves for replication. This is the lag phase, as in the manufacturing process, and requires up 2 hours and 5 min. Some organisms die before completing the lag phase because they are too damaged. The total time elapsed at this point may be 2 hours and 11 min to 2 hours and 30 min. Next comes the “log phase” in which the surviving organisms replicate, or grow logarithmically (exponentially) in great numbers. During the log phase, depending on temperature, food sources, safety, etc., the microorganisms can double in number every 10 minutes to 53 minutes. Because an adherent dental synbiotic lozenge will still be present, even after 2.5 hours, and will dissolve and become dispersed by approximately 3 hours while the user is awake, or by 8 hours during sleep, the log phase for the organisms can be reached. During this time, the organisms are protected and fed while rapidly replicating, dispersing along with the film-forming excipients, and actively colonizing and populating niches. At this point the total elapsed time is at least 3 hours (awake) to 8 hours (sleeping). Therefore, an adherent dental synbiotic lozenge could grow more probiotic organisms, more effectively than conventional dental probiotics. This is because the adherent dental synbiotic lozenge can allow the organisms to reach the logarithmic growth phase for at least 30 minutes (while the user is awake) and up to 5 hours and 30 minutes (if the user is asleep). Oral microbes grow at highly variant rates depending upon host activities—eating, doing oral hygiene, habits, sleeping, etc. To simplify, on average, oral biofilms are like a womb that can birth an average steady rate of 20.83% of their quantity of organisms every hour. Or, on the high side, while the host is sleeping, they can double their numbers every 10 minutes to every 53 minutes.

This means that an adherent synbiotic lozenge has the potential to grow many new organisms. Such a lozenge, if used while the user is awake, a minimum of 185 million new organisms can form if they multiply at an average steady rate. Or, if they grow logarithmically, they could produce greater than 2.7 billion in just 30 minutes, which is nearly as many as were in the lozenge at time of manufacture. If they replicate logarithmically, they could produce one generation (thereby doubling its organisms, or 2 to the 2^{nd} power), and up to three generations of organisms (2 to the 3^{rd} power) before totally dispersing. Thus, any organisms remaining at time 2:30 can multiply by 2 to 8 times, whereas conventional lozenges lose organisms. However, tremendous benefits can occur if used while sleeping. Since the log phase can be reached for up to 5.5 hours, and doubling can occur every 10 minutes to 53 minutes, the remaining organisms can increase dramatically. In other words, an adherent synbiotic lozenge could allow from 6.23 generations to 66 generations to progress. This rate of increase can compete with the growth of existing organisms in natural biofilm. This is why people have “morning breath”, because during sleep, salivary flow rate is low, general oral activity is reduced, and pathobiotic organisms are growing relatively freely. As such they can increase to great numbers, akin to being inside an incubator, which accounts for morning breath. Also, during sleep, is when many pathobiotics do much damage to oral structures and tissues. Thus, the ability to use sleep time to grow billions of good organisms to compete against bad organisms would be a great opportunity for improvement in oral health. And you never start with just one organism, but rather, many billions or millions. So, on the low side, if a user placed an 18-month old, expired lozenge of adherent dental symbiotic in their mouth that contained only one billion organisms at time of expiry, then about 59% of the lozenge would remain after 2.5 hours. Assuming 41% of the lozenge and all of its organisms were lost due to swallowing during the 2.5 hour lag phase, and based on how an adherent lozenge absorbs and retains moisture, softens, spreads, and forms a spreadable “supplemental biofilm” or “bio-paste”, this would translate to roughly 594 million organisms remaining that are already hydrated, grown, and are now ready to replicate.

Current art dental probiotic tablets that do not adhere intra-orally will be chewed, dissolved, and swallowed or expectorated within 30 seconds to 4 minutes. The user must actively try not to swallow the tablet ingredients too soon, which is nearly impossible. And whatever microbes are left behind will likely be washed away by saliva within 15 minutes because they don't adhere in the mouth and they haven't even had time to adapt to their new environment, let alone repair themselves and grow.

An adherent dental synbiotic lozenge is also advantageous because many probiotics are not resident strains in the oral cavity. They need all the help they can get so they can stick around, and they must be continually replenished, unless the person's interpersonal relations, locale and diet and support automatic replenishment of probiotics. (For example, a person's microbiota is malleable depending upon travel, eating different foods, interacting with new people, suffering a disease, taking antibiotics, etc.) Worse yet, pathobiotic organisms tend to reside in sticky biofilms that protect them from toxins, antibiotics, antimicrobials, and probiotics. It is unlikely that ordinary dental probiotics can be expected to ever reach their target niches and replace the pathobiotics unless they contain organisms in huge numbers are consumed and replenished constantly so that eventually some might hang on. An independent study done in 2013 showed that “dehydrated forms of probiotics may not deliver the expected dosages . . . ” and that “. . . dosages in the range of 10⁹ to 10¹⁰ colony-forming-units . . . are easily obtained by consumption of yogurt with active cultures or fermented milk products . . . ”, and that “ability to adhere to targeted sites is a desired trait”.¹ Basically, this means that with conventional dental probiotics, people may be just as well off eating yogurt.

Microbial biofilm itself is a major problem in that biofilm contains channels to provide nutrients, eliminate waste, shunt away toxins and antibiotics, protect microbe inhabitants from trauma, etc. In fact, microorganisms residing within biofilms are highly resistant (from 100 times to 1,000 times more resistant²) to antimicrobials, antibiotics, and even bacteriocins produced by probiotics. Biofilm can be penetrated by certain enzyme formulations especially designed to disrupt the biofilm matrix that embeds pathobiotics. Toxic products are often required to penetrate and kill microbes residing within biofilms. And then, if the microbes develop resistance to antibiotics and other meds, the situation becomes even worse, requiring increasingly stronger products and/or antibiotics to do the job. Microbes living within a biofilm are exponentially harder to eradicate and control than planktonic (free-floating) microbes fending for themselves in saliva. This is why mouthwashes can claim to kill “X” numbers of organisms, because it is relatively easy to kill individual “germs” that are freely floating in saliva. However, it is virtually guaranteed that most of the billions of germs living in biofilm will still be relatively intact even after vigorous oral hygiene efforts, toxic mouth rinses, and even antibiotics.

Most common dental problems (dental caries and periodontal disease) are largely preventable by following a daily regimen of excellent oral hygiene. Unfortunately, most people seem to have minimal ability, motivation, or education to achieve such a dental prevention regimen. Worse, many people have poor diets, as evidenced by the rampant spread of diabetes, obesity, intestinal problems, dental problems and other effects of poor nutrition. As a result, dental decay and periodontal disease are widespread all over the world. Also, dental caries and periodontal disease are complex, multifactorial, microbial, biofilm-related, host/pathogen/parasite, type diseases. They are not directly caused by sweets, but rather, by microbes that consume sweets as well as proteins and amino acids, and then excrete acids, inflammatory substances, and biofilm-making constituents so that they can stick within certain niches in the oral cavity and be protected while destroying oral health. As modern diets changed to include increasingly processed foods, simple carbohydrates, and sugars, the types of microorganisms inhabiting our mouths have shifted to select for entities that preferred such foods as well. Microbes that prefer simpler and sweeter carbohydrates began to colonize our mouth, thus crowding out and competing for space against other organisms that do not prefer sweets and simple carbohydrates. All microbes create their own antimicrobials (bacteriocins) and quorum signaling molecules to fight other microbes and defend their niches. Therefore, in the face of modern diets, to conquer our dental pathobiotics we need continual sources of probiotics to essentially fight fire with fire. We cannot expect antimicrobial dentifrices that become diluted by saliva, or probiotics that rarely reach effective levels to fight our battles, to address the need.

The human mouth is estimated to harbor from 700 to 1,000 different species of microorganisms. Decay and gum disease microbes have always been present in humans. Early humans exhibited some decay, but it was nowhere near the scourge it is today. It wasn't until the advent of widespread sugar consumption beginning in the 1500s that human dental decay and gum disease began to become a major problem. As our diets changed, so did the spectrum of microbes inhabiting our mouths. Our predominant pathobiotic dental microbes now are the ones that feed on our modern diets and create gooey, sticky, acidic, inflammatory substances within a protective biofilm that tends to destroy our teeth and gums. Thus, our oral pathobiotics have had plenty of time to adapt and evolve sophisticated techniques to thwart our preventive dental efforts. Yet, traditionally, to try and eradicate our dental pathobiotics, we attack them daily with broad spectrum antimicrobials, toxic products, aggressive brushing, and even antibiotics. This works temporarily in some cases, but despite all these efforts, dental diseases are still the 2^{nd} most common human affliction. Worse, in our futile attempt to kill microbes, we indiscriminately kill the good with the bad. Because the bad tend to be more hardy than the good, they recuperate sooner and repopulate faster. Furthermore, we feed the pathobiotics the foods they prefer because we eat fast foods, preserved foods, snacks, sodas, etc., so the pathobiotics regrow the soonest. In addition, we rarely eat fermented foods any more. Most foods are pasteurized and filled with preservatives and coated with insecticides, so that any probiotics are generally decimated before we can consume them. And even if they can survive, we try to kill them daily with our dental care products.

A growing number of studies have demonstrated that changes in the composition of our microbiota correlate with numerous disease or health states. When bacteria cause diseases, it is typically because they grow beyond their normal population size or niche (which can occur when the body's immune system is compromised), or because microbes enter parts of the body that are normally sterile, such as the blood, lower respiratory tract or abdominal cavity. Furthermore, relating to the germ theory of disease, we are beginning to find that most microbial diseases are not the result of just one organism acting alone. Rather, we are now understanding that most microbial diseases result from colonies and groups of organisms often living within a protective biofilm. For example, dental decay is not caused by sweets, but rather, by groups of organisms consuming various carbohydrates, and they create sticky biofilm exopolysaccharides within which they hide and reside. Microbes within a protective biofilm act differently from the same microbes freely swimming alone in a planktonic state outside of a biofilm. Microbes in biofilms can act like out-of-control mobs, but planktonic microbes freely swimming in saliva usually act like docile individuals. Thus, due to our modern western diet, and low consumption of fermented foods with live organisms, we must supplement regularly with probiotic organisms to make up for the lack of exposure to prebiotic foods and probiotic organisms.

Furthermore, the probiotic dosages in foods can vary widely, so there may not be clinically-acceptable amounts of organisms in a regular serving. Worse, we do not consume enough probiotic lozenges, and we are uncertain of the viability of cultures in the probiotic lozenges. And even when we do think we know the quantity of the cultures we ingest, the current probiotic lozenges have no substantivity to help the prebiotics survive and grow. As a result, we cannot expect to overcome our most common dental problems until we can begin recolonizing our mouths with probiotic organisms that last and take effect rather than just passing through our bodies. If we could regularly attempt to recolonize our mouths with probiotics embedded in lozenges containing prebiotic foods that the probiotics prefer, and that the pathobiotics do not prefer to use, we might be able to change the prevalence of dental diseases and shift to a default state of probiotic colonization with healthful benefits.

Accordingly, it is desired to have a lozenge that creates a prebiotic biofilm that keeps probiotics in the mouth and prevents them from merely washing down the throat. The lozenge's prebiotic residue can serve as a temporary biofilm home until the probiotic organisms can develop significant colony numbers to provide their benefits. Rather than resorting to ever-increasing toxic substances, antibiotics, and broad spectrum germ killers that simply wash away with saliva, it would be advantageous to incorporate probiotic microbes that can colonize the biofilms just like the pathobiotics can do. To this end, an adherent, long-lasting, pre/probiotic would help to reduce the use of pathobiotic biofilm fighting toxins, germicides, antibiotics, etc. It is also desirable in the art to have a means of managing, modifying, or disrupting the natural biofilm so that the probiotics can more effectively crowd out the pathobiotics and attack them with their own bacteriocins. Also needed is a means of retaining, protecting, and nourishing the probiotic organisms within the oral cavity as long as possible in order to grow the organisms and increase the effectiveness and benefits of the dose. In addition, the prebiotic nutrients should benefit the host, while being incapable of metabolizing into host-toxic byproducts.

### Definitions

A synbiotic is a probiotic/prebiotic mix that contains specific beneficial organisms and their preferred foods—foods that generally do no harm to the host and do not allow the microbes to create host-toxic metabolites.
A probiotic is a live microorganism that, when administered in adequate amounts, confers a health benefit to the host.
A prebiotic is a therapeutic nutritional preparation used for health effects favoring growth of normal bacterial flora and not favoring growth of pathobiotic or pathogenic organisms. Examples include inulin, fructooligosaccharides, arabinogalactans, non-digestible fibers, and resistant starches.
Biofilm consists of microorganisms enmeshed within a microbial-self-produced matrix of exopolysaccharides and exoproteins that strongly adheres to interfaces, resists dislodgement, and protects its component microorganisms.
Film-forming: the lozenge dissolves into a spreadable artificial or supplemental biofilm that mimics natural microbial biofilm before being swallowed.
A lozenge is a type of tablet that is designed to be retained in the mouth for an extended time.
A pathobiotic is a live organism that may or may not cause health problems for the host, as opposed to pathogens that are definitely harmful. Pathobiotics may be regular commensal human colonizers, but may not cause problems until conditions occur that are favorable to them. Such conditions may be a poor diet, diminished host immunity, systemic disease, injury, old age, and other factors.
A pathogen is an organism that definitely causes disease or health problems for the host. For example: cholera, anthrax, botulism, plague, etc.
Pharmabiotic: a combination of probiotics plus prebiotics plus postbiotics for therapeutic use.
A postbiotic is usually a non-viable microorganism-produced metabolic byproduct that has biologic activity in the host. Examples include the bacteriocin BLIS Salivaricin A, lantibiotics, short chain fatty acids, steroids, biotin, folic acid, and other vitamins. A postbiotic could also be dead probiotic cell membrane remnants containing biologic protein markers capable of causing a host response. An example related to medicinal use is killed poliovirus vaccine in which the biomarkers on the virus capsid initiates a host immune response even though the virus is dead.
Bacteriocins are proteinaceous antimicrobial peptide toxins that inhibit the growth of similar or closely related bacterial strain(s). They are produced by non-pathogenic bacteria (such as Lactobacilli) that normally colonize the human body. They may warrant serious consideration as alternatives to traditional antibiotics because these molecules exhibit significant potency against other bacteria (including antibiotic-resistant strains), are stable and can have narrow or broad activity spectra to combat infections.
Postbiotic effect is the ability of a probiotic to elicit a host response, even from nonliving organisms, and even from remnants of nonliving organisms. Postbiotics are important because some probiotics die after tableting, storage, shipping, heat, etc. Yet, despite a probiotic lozenge having lost some or many of its colony-forming units, the lozenge may still retain some effectiveness because it will contain some postbiotic antimicrobial metabolites and remnants that are capable of causing a host response.
Controlled release includes:
   Time-release: a version of metered release where the active pharmaceutical ingredient (API) is released in a steady stream of small amounts over time.
   Slow-release: the API release is delayed or slowed down, so that the lozenge does not dissolve or swell too quickly upon contact with saliva.
   Place-and-forget: the lozenge need not be manipulated, sucked on, chewed, or paid attention to, so that the user can place it and forget about it, and even safely sleep with it in place without worry of choking. This is as close to simply “taking a pill” can get.
   Extended-release: typical time-release lozenges are designed to dissolve within 30-60 minutes, but extended release lozenges can last up to 8 hours or longer, for example while sleeping

The human microbiota is the aggregate of microorganisms that resides on the surface and in deep layers of skin, (including mammary glands, saliva, oral mucosa, vagina, conjunctiva, and gastrointestinal tracts). They include bacteria, fungi, and archaea, but not micro-animals and multicellular parasites that live on the human body. Some studies estimate the total number of microbes (excluding viruses) of an average human microbiota to be greater than 100 trillion microorganisms. The average adult human body may be composed of up to 37.2 trillion cells (somatic cells+stem cells+sperm/egg cells+blood cells). This indicates that we are more microbe than human. In this respect the human body might be considered a superorganism, i.e. a communal group of microbial and human cells all working for the benefit of the collective.

The human microbiome is the genetic expression of the microbes of a microbiota living in/on a host. The total genetic material of our microbiota exceeds our own human genetic material by nearly 200× (from 100× to 360× according to the NIH and the Human Microbiome Project). Our microbiome exposes us to a vast diversity of biologically active microbial metabolites including hormones, vitamins, immunomodulators, mycotoxins, and alcohol. For example, “auto-brewery syndrome” is a rare condition wherein yeast in the intestines converts carbohydrates into ethanol which is directly absorbed into the body and produces ethanol intoxication. Therefore, our microbes can influence us in ways we are just beginning to understand. The oral region microbiota is composed of the most diverse set of microorganisms anywhere in/on the body. It is estimated that between 700 and 1,000+ different species of microorganisms live in the mouth, throat and sinuses. The oral region also contains the most diverse niches, conditions, and functions anywhere in/on the body, including:
The chronically inflamed periodontal oral-systemic interface which allows links between dental diseases and systemic diseases
Interfaces between external natural teeth and internal connective tissue
Interfaces between external artificial surfaces such as titanium implants and direct attachment to internal bone
Lips, cheeks, keratinized mucosa, non-keratinized mucosa, palate, tongue dorsum with nearly the surface area of a singles tennis court, throat, tonsils, Eustachian tubes to the middle ear, sinuses
Temporary open wounds with exposed bone after extractions
Carious holes in teeth, rotting teeth, broken teeth, loose teeth, dead teeth, abscessed teeth, endodontically treated teeth
Canker sores, bleeding gums, denture and appliance irritations, abrasions
permanently exposed dead bone as with osteonecrosis of the jaw
shedding surfaces such as skin and mucosa
non-shedding surfaces such as teeth and dental appliances
holes, pits, fissures, cracks, and leaky interfaces between tooth surfaces and restorations
Restorations with mercury or plastic resins
Crowns made of nonprecious and precious metals, ceramics and metal oxides
Prostheses made of Nylon, acrylic, and plastic
Nickel-titanium orthodontic wires and retainers
Denuded, exposed, and abraded tooth roots, receded gum margins, gingival crevices, deep gum pockets
Toxins from dentifrices, antimicrobials, smoke, vices, bad habits, infections, and microbial biofilms
Nutrients, acids, alcohol, peroxide, stomach contents (vomiting) and many other chemicals
Intimate exchanges of body fluids and microbes between others
Also, the oral environment ranges daily from extremes of moisture from fluid intake to dryness from exercise or mouth breathing

Therefore, a probiotic needs the most diverse set of microbes possible. This cannot be accomplished with conventional probiotics whereby only a few species are used and simply swallowed before they have a chance to survive. There is a need for a means of adding as many species as possible and mimicking the existing biofilm so the organisms can survive to populate their niches.

### SUMMARY

Provided is a more effective dental synbiotic lozenge that is intra-orally adhesive. The lozenge contains adhesive prebiotics and excipients, and one or more species of probiotic organisms. While safely adhered to an intra-oral surface, the lozenge dissolves to provide a controlled time-release of the excipients, prebiotics, and probiotic organisms over a period of hours instead of minutes. The lozenge dissolves into a malleable biofilm that includes a natural biofilm formed by the prebiotics and a supplemental biofilm formed by the excipients. The lozenge can be used while sleeping, which can increase probiotic availability exponentially. The result is a lozenge that allows for a smaller, but more diverse, microbial payload that can grow over time such that a fewer number of organisms are needed from the start. In one embodiment, the lozenge contains adhesive prebiotics acacia and inulin, along with additional excipients microcrystalline cellulose, HPMC K15, and sodium alginate.

### BRIEF DESCRIPTION OF THE DRAWINGS

FIG. 1 is a perspective view of an adherent lozenge in accordance with the present invention.

FIG. 2 is a side view of an adherent lozenge in accordance with the present invention.

FIG. 3 is a top view of an adherent lozenge in accordance with the present invention.

FIG. 4 is a side view of the interior of a user's mouth.

FIG. 5 is a side view of an adherent lozenge in place within the mouth.

FIG. 6 is a side view of an adherent lozenge in place within the mouth and in an intermediate stage of dissolving.

FIG. 7 is a side view of an adherent lozenge in place within the mouth and in an advanced stage of dissolving.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements, processes or techniques have been briefly mentioned and not elaborated on in order not to obscure the present invention in unnecessary detail and description. Moreover, specific details and the like may have been omitted inasmuch as such details are not deemed necessary to obtain a complete understanding of the invention, and are considered to be within the understanding of persons having ordinary skill in the relevant art.

The present invention is an adherent dental synbiotic lozenge with extended intra-oral time-release. The synbiotic lozenge is a blend of all-natural prebiotic and/or synthetic adhesive powders plus one or more probiotic strains that colonize one or more intraoral sites or niches with adequate numbers of organisms and nutrients so as to produce certain actions against pathobiotic organisms and/or provide benefits to the host human. There is no need for complex multi-layer tablets or complicated adhesive components, as it is sufficient to mix the components together and form a homogeneous lozenge with the probiotic nourishment and adhesive as part of the excipient. In fact, it is critical for safety and efficacy to make a simple lozenge with all components mixed together so that the entire lozenge can degrade into a film, instead of remaining as a discrete lozenge that could be dislodged and create a choking hazard. In addition, the lozenge includes a selection of biofilm-disrupting enzymes in some embodiments and in most embodiments it includes microbial bacteriocins—such as BLIS: Bacteriocin-Like Inhibitory Substances. These are powerful antimicrobial peptides produced only by certain strains of beneficial bacteria, which naturally form part of the normal microbial flora of the nose, mouth and throat in a healthy body. For example, the particular K12 strain of Streptococcus salivarius secretes BLIS molecules that protect against harmful bacteria responsible for strep throat, ear and upper respiratory tract infections as well as bad breath. S. salivarius is one of the most numerous beneficial bacteria found in the mouth of healthy individuals. However, only approximately 2% of people have S. salivarius with BLIS K12 activity. It would be quite beneficial to provide a sustainable source of BLIS K-12 and other bacteriocins to help prevent strep throat, middle ear infections, and so forth.

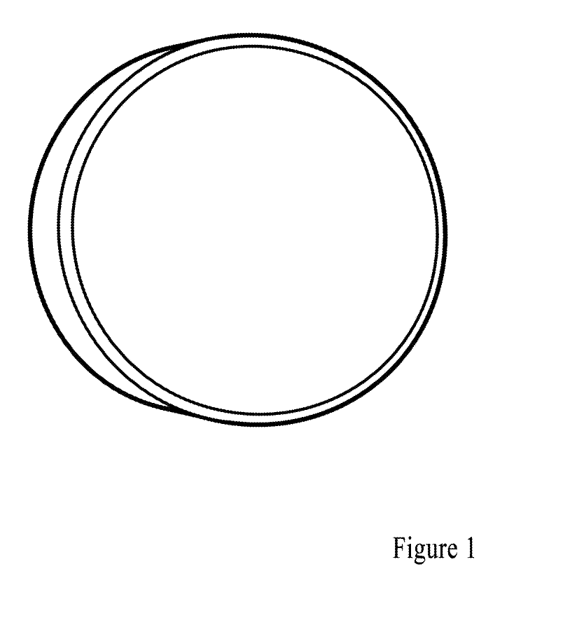

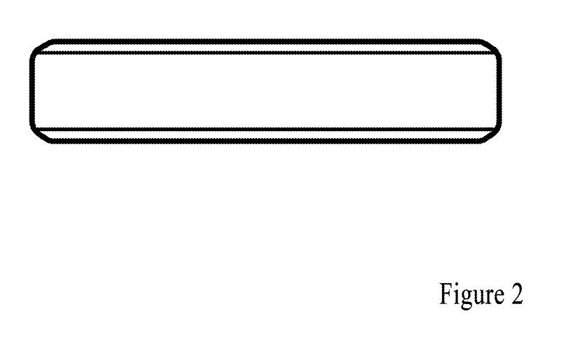

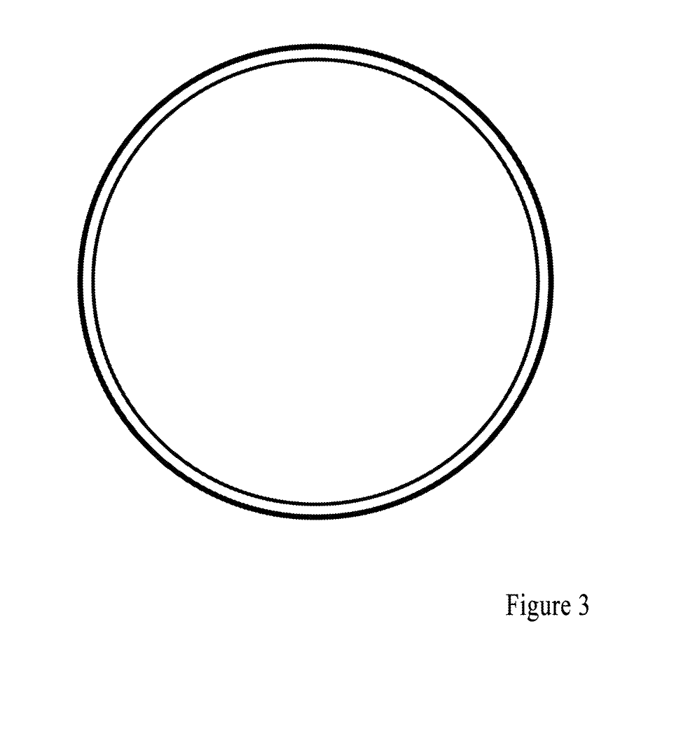

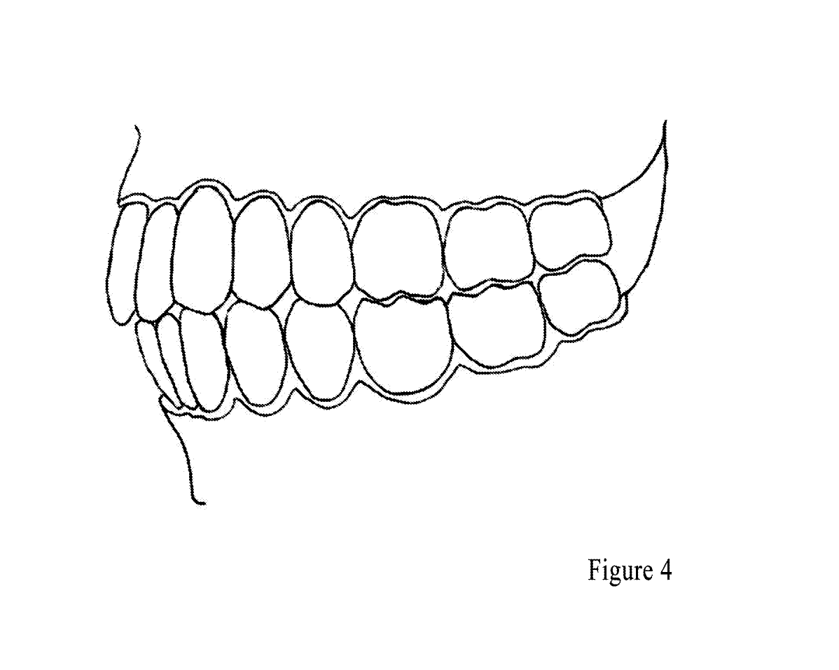

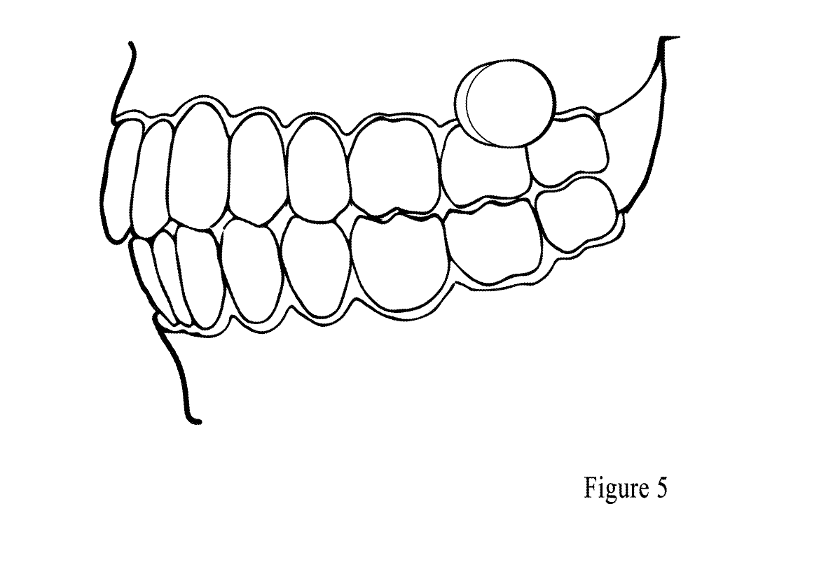

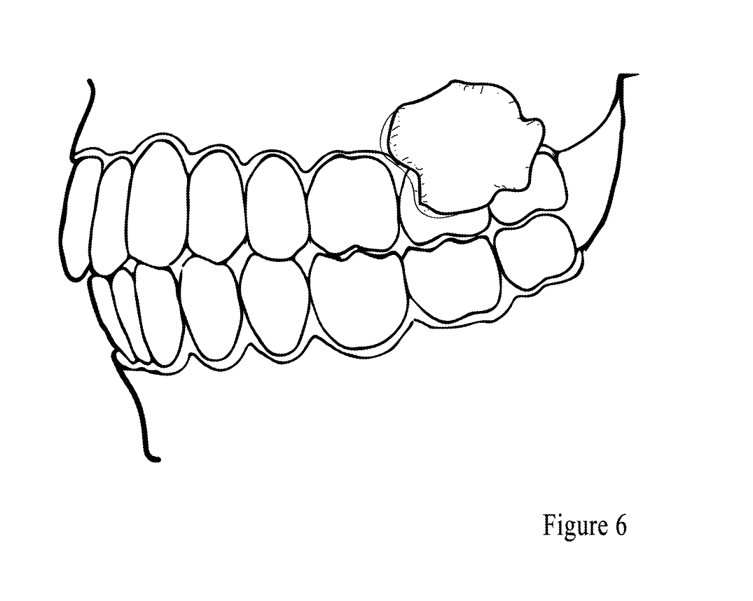

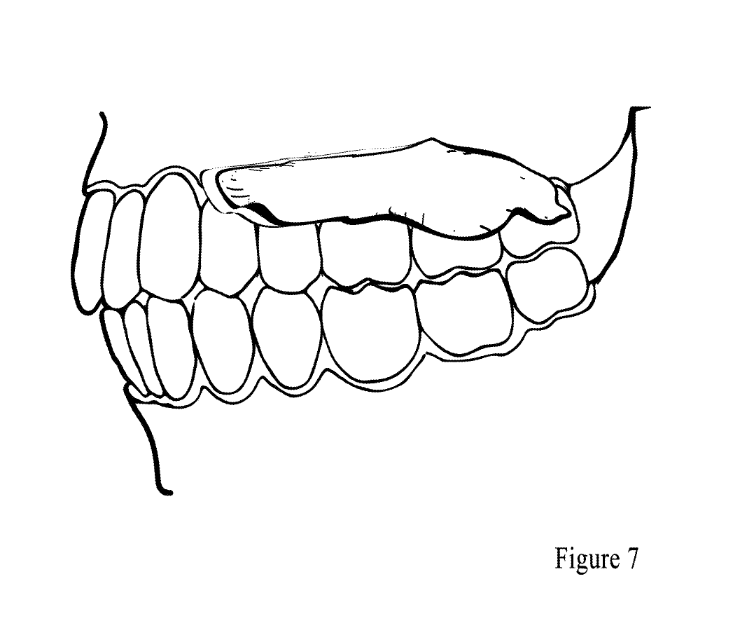

FIG. 1 is a perspective view of the adherent lozenge of the present invention. FIG. 2 and FIG. 3 are side and top views of the adherent lozenge. While the entire lozenge is adherent, it will adhere preferentially to nonmoving, stiff surfaces, leaving the movable, soft oral surfaces free of adhesion so that the exposed surfaces can dissolve. The adhesive allows the lozenge to adhere to oral tissues, teeth, crowns, dentures, braces, and other oral appliances. FIG. 4 depicts the side of a user's mouth, showing the teeth and gums. As shown in FIG. 5, the adherent lozenge is in place in the user's mouth, and more specifically, within the upper posterior cheek/gum vestibule adjacent to the upper molar. While positioning of the lozenge can be varied based on user preference, the positioning shown in FIG. 5 may be preferable to provide minimal interference from (and to) mouth movements and exposure to food and liquids. Once the user has placed the lozenge in the desired location in the mouth, they can move their mouth in various directions so that the lozenge rests in a flat/neutral position relatively unaffected by oral musculature and mouth movements. Because the entire lozenge is adhesive, there is no need to orient the lozenge in any specific way. The lozenge dissolves uniformly (as shown in FIGS. 6 and 7), and the adhesive is also a prebiotic that supports the growth of the probiotic but does not support either the growth or the harmfulness of pathobiotic organisms, and the lozenge can be easily made by the millions in conventional tablet-forming presses. Even though acacia gum is a possible prebiotic nutrient for some pathobiotic dental organisms as well as probiotic organisms, the pathobiotics do not ferment acacia into tooth-damaging metabolites. The adhesive is advantageous in that it protects, feeds and retains the organisms in the mouth as long as possible to maximize colonization of niches and population growth, helps to remineralize teeth due to calcium content, and can form a protective film around oral sores and/or protect sensitive tissues from sharp edges of braces wires, brackets, partial denture clasps, rough restorations, sharp teeth, etc. The adhesive also acts to prevent accidental swallowing of the lozenge while sleeping because once the lozenge sticks it begins eroding and softening into a pliable, soft, spreadable film instead of remaining as a discrete lozenge as shown in FIGS. 6 and 7. Moreover, the adhesive lozenge will not cause potential harm by sticking in the throat because it becomes slippery before it adheres, and the adherence time is about 5 minutes, plus, once it adheres to the teeth/gums it begins to soften and turn into a film. This is a significant safety feature that allows place-and-forget use while sleeping, which is a critical time for microbial activity.

There are several advantageous aspects of how the lozenge releases excipients and organisms over time. First, the lozenge has a delayed-release, which means that the excipients and organisms are not immediately released and adherence does not begin immediately (takes about 5 minutes). This, coupled with the fact that the lozenge initially becomes slippery in the mouth, makes it easy to swallow and eliminates the risk of becoming lodged in the throat wherein other lozenges might swell to block the airway. Second, the lozenge time-releases excipients and organisms in a metered manner so to provide a steady diffusion over time. Finally, the lozenge is extended-release in that it lasts from 3 to 8 hours and releases excipients and organisms through that time.

By incorporating the lozenge's payload into the acacia gum adhesive and viscoelastic polymers, numerous benefits occur that are superior to the current state of the art chewing gum, chewable tablets, mints, straws, powders, gels, liquids, and strips. The acacia adhesive of the present invention is a prebiotic (food for the probiotic organisms). Currently, no dental probiotics contain prebiotics that are also an oral adhesive that also protects the organisms in a place-and-forget, controlled-release film. Acacia has been shown to enhance probiotic survival during tableting, storage, shipping, and re-storage of opened bottles. Acacia has a high calcium content and has been shown to be nearly as effective at tooth remineralization as sodium fluoride, yet without the toxic connotations and worries from the “natural” community. In the mouth, acacia dissolves slowly and forms a somewhat natural biofilm that continues to protect the probiotic organisms while they are being dispersed throughout the mouth. Similarly, the excipients form an artificial biofilm which mimics the natural biofilm (dental plaque) that is nearly always present to some degree in the mouth. This artificial biofilm can coat over existing natural biofilm and smother it with probiotics delivered directly to the natural biofilm in a controlled-release fashion to overwhelm the “bad” organisms living in the natural biofilm. As used herein, the terms “artificial biofilm” and “supplemental biofilm” have the same meaning and are used interchangeably.

The adherent excipients that form the artificial biofilm also have additional beneficial properties in that they tend to lubricate the mouth, which is important for dry mouth syndrome and Sjogren's syndrome. They coat tender oral tissues, wounds, abrasions, sores, surgical sites, and exposed bone and can cover the areas of exposed bone in osteonecrosis of the jaw (ONJ), which is a very difficult condition to treat. ONJ is a challenging condition (subsequent to chemotherapy, radiation, and/or bisphosphonate use for osteoporosis) in which portions of jawbone and gums simply lose their blood perfusion and die off. This exposes dead bone which begins to harbor dangerous biofilm and pathogenic organisms, then becomes infected and the lesions spread out, sometimes causing loss of portions of the jaw. The adherent synbiotic lozenge may be able to assist with ONJ treatment and potentially save jaws and/or lives in some cases.

The mouth contains natural dental biofilm, especially in the posterior regions where sufficient brushing and flossing rarely occur. This natural biofilm harbors pathobiotics that destroy teeth and gum tissue, especially in the posterior regions, which are therefore the places where a good biofilm that can grow good microbes are needed the most. A good, artificial biofilm with its probiotics can exist on top of the bad biofilm and eventually crowd out the bad microbes if given enough time (e.g., a month), and then with consistent synbiotic use every other day/night or so, the bad biofilm could be converted to good biofilm. The good biofilm can be maintained over time based on a diet of natural foods, fermented foods, fresh fruit and vegetables, while regularly using the synbiotic to maintain desired organisms. For people with poor diets, dry mouth, genetic problems, diabetes, dementia, etc., a synbiotic supplement should be taken regularly and indefinitely.

Another benefit of artificial biofilm formation is that conventional intestinal probiotics are generally encapsulated or protected in some way so as to prevent their demise before they reach their destination(s). They need to be packaged to survive the journey through the highly acidic stomach and the bile enzymes in the small intestines. Because this journey through the stomach and small intestine is so traumatic, many of the organisms die before ever reaching their target niches. Therefore, makers of intestinal probiotics must pack many billions of organisms into the tablets so that enough might be able to survive the trip. If the same types of encapsulated or protected probiotics are applied inside the mouth, they will not be able to grow, colonize, nor populate since they are basically embalmed until they pass into the colon. However, the present invention synbiotic uses the lozenge excipients to form an artificial biofilm “womb” or “nest” within the mouth that can retain the sensitive organisms within the artificial biofilm and nourish and protect them until they can mature and survive on their own. Thus, the lozenge of the present invention differs vastly from the conventional method of probiotic encapsulation or protection.

Importantly, as a result of the lozenge protecting the organisms inside the mouth, fewer overall organisms are needed because the organisms can be grown inside the mouth, instead of having them swallowed before they can grow. Thus, fewer organisms are needed from the start. Because the lozenge requires fewer overall organisms, a greater diversity of organisms (i.e. number of different organism species) can be included within given space and cost constraints. Organism diversity is very important, and the more diversity, the better it is for the growth of good microbes and synergistic relationships among the microbes (i.e. more effective probiotic growth and health). Organism diversity is particularly important in the mouth, which has one of the most diverse micro-niches in the body. While the lozenge can include 3 billion total organisms, these billions can be made from at least 10 different organism species (identified below), whereas conventional tablets typically contain billions of a single organism or fewer than ten species). Conventional lozenges simply try to pack as many total organisms into the tablet as possible but are limited in diversity. Thus, the present invention provides a more diverse and effective synbiotic lozenge that can be manufactured and offered at an economical cost. Moreover, due to the adhesive nature of the lozenge and the unique way it dissolves and spreads, it can generate as many (or more) probiotic organisms/bacteria than were in the lozenge to begin with. In accordance with the present invention, and by way of example, even an 18-month old expired lozenge with only 1 billion viable organisms at time zero in the mouth can result in about 1.27 billion total organisms after 8 hours. And this is assuming that 41% of the lozenge and its microbes are simply swallowed or lost the in the first 2.5 hours of use, that is, before the microbes had a chance to fully repair themselves and grow enough to the point they could begin replicating themselves.

Conversely, in conventional probiotic lozenges nearly all the probiotics are swallowed and thus lost within the first 15 minutes of use in the user's mouth. Thus, due to its greater diversity and effectiveness, the lozenge of the present invention can be substantially smaller than a conventional lozenge while providing greater probiotic effect. This is significant given that consumers invariably prefer to use a smaller lozenge. In an exemplary embodiment, the lozenge can be approximately the same size as conventional lozenges, with a diameter of 11 mm and thickness of 7 mm and containing about 3 billion total organisms. However, given the superior effectiveness of the present lozenge (as described above), smaller sizes can be provided while still providing a level of effectiveness that far exceeds conventional probiotic lozenges. In an exemplary embodiment, the lozenge can have a diameter of 10 mm and thickness of 4 mm and contain about 1.4 billion total organisms. Even with only 1.4 billion organisms, the lozenge can end up with about 1.8 billion organisms after 8 hours, even after losing 41% of its organisms in the first 2.5 hours. Additionally, a smaller lozenge size designed for children may be 9 mm in diameter and 3.5 mm in thickness and contain about 1.1 billion total organisms.

Conventionally, probiotic bacteria are freeze-dried into a state of suspended animation. They need to be rehydrated, reanimated, fed, repaired, etc. Because of the adhesive property of the present lozenge, and the way in which it rehydrates, the lozenge can spread and thin out into a lenticular shape like a fried egg, wherein the central “yolk” portion is still thick reminiscent of the original lozenge shape but is substantially rehydrated. This allows the microbes residing in the “yolk” to be replicating for hours before they are released as the outer surfaces dissolve. Organisms in the center mass of the lozenge are rehydrated after about 2.5 hours and are already growing inside the remaining lozenge portion and the replicated organisms are distributed as the remaining lozenge layers keep dissolving off. This is akin to dental plaque wherein organisms are protected and continually grown and are born from the plaque at a rate of at least 20.83% of the total plaque organisms per hour. In mouth dental plaque, this 20.83% per hour equals about 4.17 billion dental plaque organisms born per hour. This results in the bad breath commonly referred to as “morning breath” which results because the bad organisms are rapidly generated and collected in the mouth due to the low saliva flow during the night that is insufficient to not wash away the organisms effectively. However, the 20.83% growth rate is an average over 24 hours. Because the growth of these bad organisms is uninterrupted overnight (e.g. by oral hygiene, drinking, eating etc.), the growth rate is substantially greater overnight during sleep hours. This is important, because the adhesive lozenge of the present invention allows the user to safely grow good probiotic organisms overnight at an accelerated rate as well, in order to compete with the plaque organisms that are born at an accelerated rate at night. Conventional probiotics do not provide this critical function.

Conventional tablets and lozenges contain excipients that usually do not protect organisms and merely dissolve and do very little for support, nutrition, and protection of organisms. Exemplary excipients for use with the synbiotic lozenge can include isomalt, inulin, Microcrystalline Cellulose, HPMC K15, Acacia seyal, sodium alginate, Glyceryl Behenate, Dicalcium Phosphate, Spearmint Flavor (natural), and Stevia. To improve the viscoelastic polymer formation within the lozenge, the following excipients can be utilized either alone or in combination: Konjac mannan and Xanthan gum, poly (glycolide-co-dl-lactide) or “PGLA,” and poly(lactic-co-glycolic acid) or “PLGA.” Inulin, Acacia seyal, Konjac mannan, and Xanthan gum are active, natural non-fermentable fibers that are prebiotic foods (i.e., food for the probiotic organisms that allows them to grow and stay alive). Inulin, Acacia seyal, Konjac mannan, and Xanthan gum also act as adhesives, and as such may be referred to herein as “adhesive prebiotics” or “natural fiber adhesives.” By providing intra-oral adhesion (to the gums, cheek, teeth, etc.) and dissolving into an adhesive gel/film, the adhesive prebiotic lozenge of the present invention prevents potential choking from accidental swallowing of the lozenge (e.g., during overnight use).

Microcrystalline Cellulose, HPMC K15, and sodium alginate are excipients that can be considered “artificial” adhesives, and provide film-forming and spreading of the lozenge. These excipients are also provide the lozenge with its dissolvable, time-release characteristics. The remaining excipients in the lozenge act to keep it together, prevent crumbling, make the tableting process run smoothly, keep the machines from jamming, flavoring, etc. The excipients are chosen for their ability to:
adhere to oral tissues (teeth and/or gums) and dental appliances (dentures, partial dentures, braces)
dissolve slowly over 3 to 8 hours by morphing from a lozenge shape to a pasty film within the first 15 minutes and continue forming a film for 3-8 hours.
form a film to maintain adherence
form a film-like artificial biofilm (i.e. supplemental biofilm) around the organisms to protect them inside the mouth to facilitate rehydration of the organisms and facilitate adaptation of the organisms to their new environment (lag phase)
feed the organisms during their logarithmic growth phase (log phase)
help the organisms to spread out and colonize and populate their respective niches (teeth, gums, under the gums, throat, tonsils, sinuses, etc.), including colonization of niches
protect the organisms as they spread out with the artificial biofilm (i.e. supplemental biofilm)
remineralize teeth in the process (dicalcium phosphate and acacia) and provide the ability to add more minerals to the teeth surfaces and repair decalcified regions
provide time-release, extended-release, and slow-release of the organisms as well as the remineralizing excipients
create a safety feature by having the lozenge dissolve into a spreadable film that avoids a potential choking hazard
the film-forming safety allows the use of the lozenge while sleeping, which is the most opportune time to use probiotics

In an exemplary embodiment, the adhesive lozenge has the following composition: 150-200 mg of isomalt, 60-80 mg of inulin, 70-125 mg of microcrystalline cellulose, 75 mg of HPMC K15, 40-80 mg of Acacia seyal, 20 mg of sodium alginate, 10 mg of glyceryl behenate, 10 mg of dicalcium phosphate, 10 mg of natural spearmint flavor, and 1 mg of Stevia. This lozenge composition can be adjusted accordingly based on the desired lozenge size (i.e. volume). In a preferred embodiment, adhesive lozenge contains 150 mg of isomalt, 80 mg of inulin, 80 mg of microcrystalline cellulose, 75 mg of HPMC K15, 60 mg of Acacia seyal, 20 mg of sodium alginate, 10 mg of glyceryl behenate, 10 mg of dicalcium phosphate, 10 mg of natural spearmint flavor, and 1 mg of Stevia. In an exemplary embodiment, the prebiotics inulin and Acacia seyal are replaced with comparable amounts of Konjac mannan and Xanthan gum.

Conventional probiotic tablets on the market tend to either dissolve too quickly, or when they dissolve, they feel gritty and have an undesirable feeling in the mouth due to the tableting products and possible encapsulation process for the probiotics. This is because conventional excipients add a bothersome “gritty” feel or chalky feel and taste that turns people off. This is avoided by the lozenge of the present invention which incorporates the probiotics within the Acacia adhesive and completely surrounds all aspects in soluble adhesive. Acacia has no taste or gritty feel during dissolution. The problem with current art tablets are numerous, especially because they do not mimic the life of an actual organism in a biofilm.

The acacia remineralization factor is important because some of the probiotic organisms excrete lactic acid, which has potential to demineralize teeth. Since acacia is a microbial food, and it forms an adhesive gel-like substance with water, many of the probiotics are held within the gel as it disperses through the mouth and sticks to oral tissues, teeth. Acacia can act as a prebiotic biofilm, in that it can stick to oral surfaces, harbor and feed many of the probiotic organisms, allow them to grow and colonize, and as the acacia gel slowly dissolves away it can protect and carry some of the planktonic free-floating organisms so as to favor niche colonization. Current probiotic technology usually does not use a prebiotic, nor an adhesive to enhance the ability of organisms to stick to their niches. Current products result in the probiotics merely swishing temporarily in the mouth before being swallowed. Current technology does not mimic how microbes form plaque biofilm and colonize the mouth. The present invention mimics the natural state of biofilm formation, protection of organisms until they can colonize, and prevents the organisms from merely being swallowed after 5-15 minutes. Thus, the lozenge provides a safe haven for the microbes to rehydrate, acclimatize, adapt, and grow unmolested by salivary enzymes, immunoglobulins, and other agents. Conventional probiotic tablets and lozenges are akin to mints that dissolve within 5-15 minutes, or chewables that are swallowed within a minute, which drastically reduces their effectiveness.

Additional uses for the synbiotic lozenge include chewing it and using it as a dentifrice if desired. Because it forms a slippery film, it can facilitate flossing between tight teeth. In this way, lozenge ingredients can be forced directly into the gum crevices to speed up colonization of periodontal niches. Also, chewing a lozenge just before bedtime could embed the probiotics into teeth grooves, pits, fissures, cracks, leaky fillings, restoration margins, and so forth for extra protection of teeth until the user can get to a dentist or obtain a crown or new filling.

Most people eat a meal, snack, etc. and just leave the food residue on their teeth until maybe they brush at night before going to bed. Because of inadequate or total lack of brushing, food residue and fermenting biofilm often reside unmolested on teeth (especially posterior teeth) for days and weeks, or even until a 6-month dental cleaning. Most of the time, this food residue is from easy, simple, processed, fast foods that are full of sugars and simple carbohydrates that actually are the preferred foods of pathobiotics. This means that food for decay and gum disease microbes is always present. If we were to radically change our diet to eating mostly raw and fresh fruits and vegetables, complex carbs, resistant starches, insoluble fibers, and fermented foods, much like we did many thousands of years ago before the advent of fast foods, preservatives, and refrigeration, we would have far fewer dental problems. This is because probiotic organisms favor our old-fashioned foods, whereas pathobiotics favor our current foods. Unfortunately, due to our busy lifestyles, need for preservatives and processed, pasteurized foods, shipping, storage, easy preparation, desire for sweets, etc., we have gradually selected for the growth of pathobiotic dental organisms.

Current art probiotics do not mimic this “sticky” factor. Probiotic organisms prefer certain prebiotic foods that are not the same foods that pathobiotic organisms like to eat. Probiotics like to eat indigestible fibers, fructo-oligo-saccharides, resistant starches, larger chain carbohydrates and fatty acids. Pathobiotics prefer simpler carbs and food sources. Thus, using a current art probiotic merely puts some organisms into the mouth but doesn't feed, protect, nor support them with their own preferred food type. For example, a person could easily starve in a jungle, despite being surrounded by “food,” if they are unable catch it, prepare it, and cook the food for consumption. The same is true for probiotics.

The adhesive lozenge should preferably be placed on the upper buccal alveolar mucosa superior to the 1st and 2nd molars because that is directly opposite from the parotid gland in the cheek, which provides copious saliva flow to dissolve the lozenge and distribute the payload throughout the mouth, while also tending to buffer and neutralize any acids that might result from microbial metabolic activity. Without an adhesive to hold the lozenge on an oral surface (e.g. gums, teeth, crowns, dentures, braces, or other oral appliances) an ordinary tablet would tend to slide around and become annoying, while also becoming lodged on teeth surfaces, which might result in demineralization instead of on mucosal surfaces which do not demineralize. Because the lozenge is adhesive it can be used at night while sleeping. In the mouth, night time is the best time to grow organisms because the saliva flow rate is reduced, dilution is reduced, the mouth is moist and warm like an incubator, and as a result, billions of pathobiotics grow uninhibited overnight (i.e. “morning breath”). Therefore, the best time to grow probiotics is also overnight, and by using the adherent synbiotic of the present invention, there is virtually no risk of accidentally swallowing or inhaling it while sleeping. In addition, the adherent synbiotic can remain in place while talking, working, exercising, at parties, and so forth. In this regard it provides safety from accidental choking or inhalation of the lozenge. As a result, the user can quickly place the lozenge in the mouth, and once adhered to the desired location, the user can forget about the lozenge and go about their normal activities while the lozenge slowly dissolves into a film over several hours. If the user does not like the position of the lozenge within the mouth, it can be easily re-positioned to a new location within the first 30-minutes of use (before it begins transitioning to gel form) and will adhere to that new location. Conventional adhesive lozenges do not have the advantage of being re-positionable because re-positioning would typically break the bond between the active ingredient (e.g. xylitol) and the adhesive.

The payload mix is also important because certain organisms work with synergy. Most conventional dental probiotics consist of just one or a few species of organisms. The synbiotic lozenge of the present invention contains at least 10 organisms that together act to help prevent conditions such as oral cancer, plaque formation, decay, Strep throat, sinus infection, otitis media, gingivitis, periodontal disease, bad breath, and oral infections. Given the link between oral and systemic health, improving oral health in this manner also has the potential to alleviate systemic problems such as heart disease, stroke, pneumonia, Alzheimer's, erectile dysfunction, diabetes, and other orally/systemically-linked diseases that are being discovered yearly. An exemplary set of 10 organisms for use in the synbiotic lozenge of the present invention are identified below with their specific target areas and benefits:
1. Lactobacillus acidophilus
   Oral, vaginal, intestinal
   Seems to reduce incidence of Streptococcus mutans (the major dental decay bacterium), controls Candida albicans, inhibits E. coli, inhibits Helicobacter pylori (stomach ulcers), inhibits Salmonella (food poisoning), inhibits Shigella (diarrhea), inhibits Staphylococcus, produces many antimicrobials and enzymes
2. Lactobacillus casei
   Targets the gums, inhibits S. mutans (dental caries), inhibits S. sobrinus (dental caries), reduces C-reactive protein, reduces Irritable Bowel Syndrome, reduces inflammation
3. Lactobacillus paracasei
   Targets periodontal disease, binds to Porphyromonas gingivalis (gum disease)—(by the way, P. gingivalis is rarely detected in people who do not have gum disease)
4. Lactobacillus salivarius
   Targets the gums, inhibits Tannerella forsythia (gum disease), inhibits Porphyromonas gingivalis, works in tandem with Lactobacillus plantarum, inhibits S. mutans, reduces caries, reduces strep throat, inhibits Strep pyogenes (strep throat), reduces ulcerative colitis, inhibits Candida, inhibits Salmonella
5. Lactobacillus plantarum
   Targets periodontal disease, works in tandem with L. salivarius, reduces Irritable Bowel Syndrome, reduces Interleukin 6 levels—which may affect perio disease
6. Lactobacillus reuteri
   Targets the gums, reduces gingivitis, reduces caries, significantly lowers S. mutans counts, secretes reuterin and other bacteriocins, reduces inflammation by reducing pro-inflammatory cytokines, works similar to L. acidophilus, works also on gastrointestinal membranes, colonizes mouth, nasal cavity, pharynx, stomach, duodenum, adheres to host tissues, and has many other functions
7. Lactobacillus rhamnosus GG
   Targets periodontal disease, reduces Candida, reduces caries, inhibits S. mutans, inhibits S. sobrinus, inhibits inflammation
8. Lactobacillus helveticus
9. Streptococcus thermophilus
   Targets decay, enters biofilm on hydroxyapatite, interferes with Strep sobrinus, affects Strep mutans, reduces acute diarrhea rotavirus, reduces intestinal permeability, reduces Helicobacter pylori (stomach ulcers)
10. Streptococcus salivarius K-12
   Freshens breath, reduces sinusitis, fights Strep pyogenes (strep throat), reduces ulcerative colitis
11. Streptococcus salivarius M-18
   Fights decay, breaks up dental plaque, releases urease, neutralizes plaque acids

In an exemplary embodiment, the lozenge contains one or more of the aforementioned species of probiotic organisms. In a preferred embodiment, the lozenge contains all 11 of these species, i.e. Lactobacillus acidophilus, Lactobacillus casei, Lactobacillus paracasei, Lactobacillus salivarius, Lactobacillus plantarum, Lactobacillus reuteri, Lactobacillus rhamnosus GG, Lactobacillus helveticus, Streptococcus thermophilus, Streptococcus salivarius K-12, and Streptococcus salivarius M-18. In one embodiment, the total organism count in the lozenge is 3 billion or less without no more than 300 million organisms of each species.

The advantages of the probiotic lozenge of the present invention are numerous. First, adhesion for 3 to 8 hours to oral tissues, structures and dental appliances, which provides a place-and-forget functionality. One unexpected, yet very fortunate result of the synbiotic ingredient formulation is that the lozenge does not remain as a discrete object in the mouth. Rather, it degrades fairly quickly into a slippery film that spreads out. This is a critical safety feature because an orally-retained lozenge could dislodge during sleep and cause a choking hazard. The lozenge formulation is also unique in that the formulation allows millions of lozenges to be produced economically and efficiently, without manufacturing difficulties (i.e. unwanted sticking to the manufacturing presses and resultant jamming of machinery).

One of the goals of microbial balance is to keep the bad microbes to 15% or less of the total microbiome. The dental probiotic lozenge in accordance with the present invention can still have 59% of a lozenge remaining (i.e. 41% dissolved away) after 2.5 hours of use, which means there are at least 1.78 billion microbes ready to replicate. If those organisms replicate at merely a linear rate (and not an exponential rate as is common), those 1.78 billion organisms could grow an additional 2.038 billion more organisms in 5.5 hours, bring the total organisms remaining plus the ones grown to 3.82 billion. This is 127% of the microbes in the original lozenge. By way of example, one can look at a person that is on the borderline of disease with exactly 15% of total organisms and pathobiotics. Assuming their dental plaque contains 20 billion organisms and grows at a rate of 100 billion organisms per 24 hours, then 15% of 120 billion total organisms=18 billion pathobiotics. So, by merely adding 3.82 billion probiotic organisms, to the 120 billion yields 123.82 billion cells, and the 18 billion pathobiotics becomes 14.54% instead of 15%.

Therefore, if a person is at the critical point of 15% pathobiotics, the additional 3.8 billion microbes grown overnight could reduce the pathobiotic count to about 14.54% of the overall population, thus keeping the person out of danger. This is in addition to whatever host benefits the probiotics provide by simply being there and actively fighting pathobiotics. Such performance and benefit is not offered by conventional probiotics. Even if using a probiotic lozenge in accordance with the present invention that has recently expired, the calculations demonstrate that the dental plaque will still have fewer than 14.84% pathobiotics.

The 80/20 Pareto Principle, which has been shown to be valid in many fields of study, states that 80% of effects are due to 20% of the causes. The Pareto Principle can be further modified by taking 80% of the 80% and 20% of the 20% to obtain another principle that states 64% of effects occur from 4% of the causes, and taking it another iteration, that 51.2% of effects are due to 0.8% of causes. Extrapolating the Pareto Principle to oral microbes, by focusing on controlling only 0.8% to 4% of the worst offenders, oral health can be dramatically improved. When pathobiotics exceed 15% of the total oral microbiome, dental problems can occur, which is roughly in line with the Pareto Principle. The mouth contains about 1,000 species of microbes. Despite the billions of different microbes, we know only a few of them in much detail. Most of the microbes are transient and indifferent, and do not seem to cause any problems. Many are indigenous strains that we acquired in the first 3 years of life and became established with us as we grew, developed habits, diets, lifestyles, pets, geographical locations, societies, families, friends, etc. Some are probiotics and some are pathobiotics. A few of the pathobiotics are particularly bad—Streptococcus mutans (tooth decay), Porphyromonas gingivalis and Fusobacterium nucleatum (periodontal disease), Streptococcus pyogenes (strep throat). This means that 0.8% to 4% of the oral microbes can cause 51.2% to 64% of the problems. Therefore, by applying the 64/4 and the 51.2/0.8 derivatives of the Pareto Principle, the effect of controlling between 0.8% and 4% of microbes can dramatically reduce the potential for disease.

In a preferred embodiment, as set forth above, the lozenge contains 10 probiotic strains that target the most pathobiotic organisms, while also creating enough overall growth to keep the total number of all pathobiotics below 15%. Even if a lozenge did not grow enough total probiotics to keep the pathobiotics below 15%, because of the specificity of the probiotics to target the worst offenders, it can control between 0.8% and 4% of the pathobiotics. This can result in dramatically limiting up to 64% of dental problems. Thus, the lozenge of the present invention has a dual capability in that it is able to grow enough good bacteria to keep bad organisms below 15%, and it targets the most damaging 0.8% to 4% of pathobiotics that cause between 51.2% and 64% of most common dental problems.

Furthermore, the prebiotic fibers inulin and acacia provide probiotic food, support, protection, and growth for the organisms, and acacia promotes re-mineralization of tooth enamel. The mix of HPMC, sodium alginate, acacia, and inulin provides several advantageous functions. First, it provides for smooth and trouble-free tableting machinery operation. Second, it provides intraoral slipperiness and delayed adhesion to prevent premature adhesion in case the lozenge is accidentally inhaled or swallowed—this is actually a safety feature because pills or tablets can sometimes get stuck in the throat and cause swelling, and if the lozenge contains an agent that adheres quickly and swells, the choking risk is compounded. Therefore, a slippery lozenge is an advantage to facilitate passage through the esophagus in case of swallowing. Finally, it provides an artificial or supplemental biofilm formation. This protects and nourishes the probiotics and prevents them from being swallowed prematurely. As the artificial biofilm spreads, it protects and carries organisms with it, rather than letting them fend for themselves in a hostile environment. The artificial biofilm can cover existing biofilms and help deliver probiotics directly into the biofilms so they can compete with pathobiotics. An unexpected advantage of film formation is the ability to cover oral wounds, ulcers, surgery sites, and extraction sockets. Another aspect of film formation is the potential to cover exposed bony lesions of osteonecrosis of the jaw (ONJ). This could be a life-saving feature.

Fewer total numbers of organisms can be used since the organisms can actually grow to greater numbers than were originally packed into each lozenge. As a result, a greater diversity of organisms can be included in each lozenge, which means more opportunity to colonize the vast number of oral niches. The lozenge is more economical, less wasteful, more socially responsible than current dental probiotics. Finally, the lozenge can be used as a dentifrice if it is chewed and flossed between teeth and brushed around teeth. By utilizing the optimal probiotics in the manner described, the lozenge advantageously departs from conventional reliance on toxic broad-spectrum oral antimicrobials, antiseptics, antibiotics, and thus reduces the potential for antibiotic-resistant strains, allergies, and potentially break some of the links in the oral-systemic chain of diseases, such as strokes, cardiac problems, and dementia.

While there have been described herein what are considered to be preferred and exemplary embodiments of the present invention, other modifications of the invention shall be apparent to those skilled in the art from the teachings herein. It is noted that the embodiments disclosed are illustrative rather than limiting in nature and that a wide range of variations, modifications, changes, substitutions are contemplated in the foregoing disclosure and, in some instances, some features of the present invention may be employed without a corresponding use of other features. Many such variations and modifications may be considered desirable by those skilled in the art based upon a review of the foregoing description of preferred embodiments.

### ENDNOTES

¹Banas J A, Popp E T. Recovery of Viable Bacteria from Probiotic Products that Target Oral Health. Probiotics and antimicrobial proteins. 2013; 5(3): 227-231. doi:10.1007/s12602-013-9142-2 .
²Biofilm formation mechanisms and targets for developing antibiofilm agents. Future Medicinal Chemistry; Vol. 7, No. 4, Pages 493-512. DOI 10.4155/fmc.15.6; Nira Rabin, Yue Zheng, Clement Opoku-Temeng, Yixuan Du, Eric Bonsu & Herman O Sintim.

What is claimed is:

1. An adherent dental synbiotic lozenge comprising:
one or more prebiotics, including at least one adhesive prebiotic configured to provide intra-oral adhesion of the lozenge;
a plurality of excipients, including at least one adhesive excipient selected from the group: microcrystalline cellulose, HPMC K15, sodium alginate;
one or more species of probiotic organisms;
wherein the lozenge dissolves to provide a controlled time-release of the excipients, prebiotics, and probiotic organisms; and
wherein the lozenge dissolves into a malleable biofilm, wherein the malleable biofilm further comprises a natural biofilm formed by the prebiotics and a supplemental biofilm formed by the excipients.

2. The lozenge of claim 1 wherein the at least one adhesive prebiotic is selected from the following group: inulin, Acacia seyal, Konjac mannan, Xanthan gum.

3. The lozenge of claim 1 wherein the at least one adhesive prebiotic includes inulin and Acacia seyal.

4. The lozenge of claim 1 wherein the at least one adhesive prebiotic includes Konjac mannan and Xanthan gum.

5. The lozenge of claim 1 further comprising one or more of the following excipients: isomalt, glyceryl behenate, dicalcium phosphate, natural spearmint flavor, Stevia.

6. The lozenge of claim 1 wherein the species of probiotic organisms includes one or more species from the following group: Lactobacillus acidophilus, Lactobacillus casei, Lactobacillus paracasei, Lactobacillus salivarius, Lactobacillus plantarum, Lactobacillus reuteri, Lactobacillus rhamnosus GG, Lactobacillus helveticus, Streptococcus thermophilus, Streptococcus salivarius K-12, Streptococcus salivarius M-18.

7. The lozenge of claim 1 having a total organism count of 3 billion or less.

8. The lozenge of claim 1 wherein each species of probiotic organism includes no more than 300 million organisms.

9. The lozenge of claim 1 wherein the intra-oral adhesion occurs after approximately 5 minutes of placement in a user's mouth.

10. The lozenge of claim 1 wherein the controlled time-release of the excipients, prebiotics, and probiotic organisms occurs after approximately five minutes of placement in a user's mouth.

11. The lozenge of claim 1 wherein the controlled time-release of the excipients, prebiotics, and probiotic organisms occurs over a period of 3 to 8 hours.

12. The lozenge of claim 1 having intra-oral adhesion such that the lozenge adheres to the gums, cheek, teeth, crowns, dentures, braces, or other oral appliances.

13. The lozenge of claim 1 containing the following components: 150-200 mg of isomalt, 60-80 mg of inulin, 70-125 mg of microcrystalline cellulose, 75 mg of HPMC K15, 40-80 mg of Acacia seyal, 20 mg of sodium alginate, 10 mg of glyceryl behenate, 10 mg of dicalcium phosphate, 10 mg of natural spearmint flavor, and 1 mg of Stevia.

14. An adherent dental synbiotic lozenge comprising:
one or more prebiotics configured to provide intra-oral adhesion of the lozenge, said prebiotics including inulin and Acacia seyal;
one or more excipients, said excipients including isomalt, microcrystalline cellulose, HPMC K15, sodium alginate, glyceryl behenate, dicalcium phosphate, natural spearmint flavor, and Stevia;
one or more probiotic organism species selected from the following group: Lactobacillus acidophilus, Lactobacillus casei, Lactobacillus paracasei, Lactobacillus salivarius, Lactobacillus plantarum, Lactobacillus reuteri, Lactobacillus rhamnosus GG, Lactobacillus helveticus, Streptococcus thermophiles, Streptococcus salivarius K-12, Streptococcus salivarius M-18.
wherein the lozenge dissolves to provide a controlled time-release of the excipients, prebiotics, and probiotic organisms; and
wherein the lozenge dissolves into a malleable biofilm, wherein the malleable biofilm further comprises a natural biofilm formed by the acacia gum and a supplemental biofilm formed by the excipients.

15. The lozenge of claim 14 containing the following components: 150-200 mg of isomalt, 60-80 mg of inulin, 70-125 mg of microcrystalline cellulose, 75 mg of HPMC K15, 40-80 mg of Acacia seyal, 20 mg of sodium alginate, 10 mg of glyceryl behenate, 10 mg of dicalcium phosphate, 10 mg of natural spearmint flavor, and 1 mg of Stevia.
